(19) United States
(12) Patent Application Publication
(10) Pub. No.: US 2017/0189446 A1
(43) Pub. Date: Jul. 6, 2017

(54) Emulsion of Carotenoids and Ocular Antioxidants

(71) Applicants: Joyce Hendler, (US); Guardion Health Sciences, LLC, San Diego, CA (US)

(72) Inventor: Sheldon Hendler, La Jolla, CA (US)

(21) Appl. No.: 15/260,827

(22) Filed: Sep. 9, 2016

Related U.S. Application Data
(63) Continuation of application No. 14/028,104, filed on Sep. 16, 2013.
(60) Provisional application No. 61/701,482, filed on Sep. 14, 2012.

(51) Int. Cl. A61K 33/34; A61K 47/36; A61K 47/24; A61K 36/45; A61K 31/385; A61K 47/26; A61K 47/10; A61K 47/12; A61K 31/355; A61K 31/593; A61K 31/015; A61K 31/353; A61K 31/122; A61K 9/107; A61K 31/51; A61K 31/375; A61K 33/04; A61K 33/06; A61K 33/32; A61K 33/00; A61K 31/198; A61K 31/145; A61K 31/197; A61K 31/525; A61K 31/455; A61K 31/675; A61K 31/555; A61K 31/4188; A23L 33/105; A23L 33/15; A23L 33/16; A61K 31/047

(52) U.S. Cl. CPC A61K 33/34; A61K 31/047; A61K 47/36; A61K 47/24; A61K 36/45; A61K 31/385; A61K 47/26; A61K 47/10; A61K 47/12; A61K 31/355; A61K 31/593; A61K 31/015; A61K 31/353; A61K 31/122; A61K 9/107; A61K 31/51; A61K 31/375; A61K 33/04; A61K 33/06; A61K 33/32; A61K 33/00; A61K 31/198; A61K 31/145; A61K 31/197; A61K 31/525; A61K 31/455; A61K 31/675; A61K 31/555; A61K 31/4188; A23L 33/105; A23L 33/15; A23L 33/16; A23V 2002/00

(57) ABSTRACT

A daily liquid supplement for ocular and body health containing at least one of lutein, zeaxanthin, meso-zeaxanthin and astaxanthin for a human subject for nutritionally supplementing macular pigments is disclosed. The micronized nutrients in a lipid based emulsion are more efficiently absorbed into the bloodstream than conventional supplement formulations resulting in higher serum levels and increased macular pigment.

### CROSS-REFERENCES

This application is a continuation of non-provisional U.S. patent application Ser. No. 14/028,104, filed Sep. 16, 2013, which claims the benefit of priority from U.S. Provisional Application No. 61/701,482, filed Sep. 14, 2012, the entire contents of which are incorporated herein by reference.

### FIELD OF THE INVENTION

The Invention relates to nutritional supplements for enhancing assayable macular pigment levels. More particularly, the Invention relates to aqueous based emulsions of carotenoids in combination with ocular antioxidants.

### BACKGROUND OF THE INVENTION

The macula is an anatomical feature of the eye located near the center of the retina. The macula is characterized by a high concentration of cone cells and of macular pigments. The center of the macula is called the fovea, which is encircled by the parafovea and perifovea. The fovea contains the largest concentration of cone cells and is responsible for central, high resolution color vision. (Hirsch, J., et al., 1989, Vision Research, 1989, vol. 29, pp 1095-1101) The parafovea and perifovea, respectively, have lesser concentrations of cones.

Macular pigments impart a yellow color to the macula. The principle macular pigments are xanthophyll type carotenoids, viz., lutein, zeaxanthin, and meso-zeaxanthin. (Bone, R. A., et al., 1997, Experimental Eye Research, vol. 64, pp 211-218; Johnson, E. J., et al., 2005, Investigative Ophthalmology & Visual Science, vol. 46, pp 692-702) The distribution of macular pigments across the macula is not uniform. Zeaxanthin and meso-zeaxanthin predominate in the fovea; lutein predominates in the parafovea. (Bone, R. A., et al., 1988, Investigative Ophthalmology & Visual Science, vol. 29, pp 843-849; Snodderly, D. M., et al., 1991, Investigative Ophthalmology & Visual Science, vol. 32, pp 268-279) The concentration of meso-zeaxanthin peaks centrally. (Bone et al., 1997, supra)

Macular pigment protects the macula by filtering damaging blue and near-ultraviolet light. (Snodderly, D. M., et al., 1984, Investigative Ophthalmology & Visual Science, vol. 25, 660-673) Macular pigment also protects the macula by serving as a powerful antioxidant. (Khachik, F., et al., 1997, Investigative Ophthalmology & Visual Science, vol. 38, pp 1802-1811) The level of macular pigment as measure by optical density varies significantly from one individual to the next. (Hammond, B. R., et al., 1997, Journal of the Optical Society of America A: Optics, Image Science, vol. 14, pp 1187-1196) Also, some individuals have an atypical distribution of macular pigments, i.e., they lack a typical central peak of mesozeaxanthin. (Berendschot, T. T. J. M., et al., 2006, Investigative Ophthalmology & Visual Science, vol. 47, pp 709-714; Delori, F. C., et al., 2006, Journal of the Optical Society of America A: Optics, Image Science, vol. 23, pp 521-538) Such atypical macular pigment distribution adversely impacts the protective role of macular pigment and Increases the risk of developing age related macular degeneration. (Trieschmann, M., et al., 2003, Graefes Archive for Clinical and Experimental Ophthalmology, vol. 241, pp 1006-1012) Fortunately, atypical macular pigment distribution may be corrected by dietary supplementation with carotenoids. (Nolan, J. M., et al., 2012, Experimental Eye Research, 2012, vol. 101, pp 9-15) Additionally, there is evidence that macular pigments, and by Implication dietary carotenoid supplementation, may enhance visual performance and comfort. (Bartlett, H. E., et al., 2008, Clinical Nutrition, vol. 27, pp 218-227; Engles, M., et al., 2007, Investigative Ophthalmology & Visual Science, vol. 48, pp 2922-2931; Hammond Jr., et al., 2005, Ophthalmic and Physiological Optics, vol. 25, pp 315-319; Kvansakul, J., et al., 2006, Ophthalmic and Physiological Optics, vol. 26, pp 362-371; Loughman, J., et al., 2010, Vision Research, vol. 50, pp 1249-1256; Nolan, J. M., et al., 2011, Vision Research, vol. 51, pp 459-469; Rodriguez-Carmona, M., et al., 2006, Ophthalmic and Physiological Optics, vol. 26, pp 137-147; Stringham, J. M., et al., 2007, Optometry & Vision Science, vol. 84, pp 859-864; Stringham, J. M., et al., 2008, Optometry & Vision Science, vol. 85, pp 82-88; Stringham, J. M., et al., 2004, Investigative Ophthalmology & Visual Science, vol. 45, pp 3838-3848; and Wooten, B. R., et al., 2002, Progress in Retinal and Eye Research, vol. 21, pp 225-240)

Age-related macular degeneration, a condition correlated with depleted macular pigments, is presently the most common cause of blindness in first world countries. (Klaver, C. C. W., et al., 1998, Archives of Ophthalmology, vol. 116, pp 653-658). To address this problem, the National Eye Institute (USA) conducted a major study to characterize the specific role of dietary lutein and zeaxanthin in combination with antioxidants, vitamins, minerals, and omega fatty acids in the progression and advancement of age-related macular degeneration, i.e., the Age-Related Eye Disease Study 2 (AREDS2). Participants of the study took one of four AREDS formulations daily for five years (AREDS2 Research Group, May 5, 2013, JAMA, published online). The results showed that participants who took an AREDS formulation with lutein and zeaxanthin but no beta-carotene, reduced their risk of developing AMD over the five years by about 18 percent compared to the participants who took an AREDS formulation with beta-carotene but no lutein or zeaxanthin. Additionally, participants with low dietary intake of lutein and zeaxanthin before the study, who took the formulation containing lutein and zeaxanthin, showed a 25% decrease in likelihood of developing AMD compared to participants with the same dietary intake who did not receive lutein and zeaxanthin. A report published in Ophthalmology by the AREDS Research Group showed the beneficial effects of taking AREDS vitamins are long-lasting (Chew et al., Apr. 11, 2013, Ophthalmology, published online).

Whereas lutein and zeaxanthin are derived from diet, meso-zeaxanthin, in unsupplemented individuals, is generated in situ from the isomerization of macular lutein. (Johnson et al., 2005, supra) Meso-zeaxanthin accounts for about a third of total macular pigment, in spite of its absence from conventional diets. (Bone, R. A., et al., 1993, Investigative Ophthalmology & Visual Science, vol. 34, pp 2033-2040) Lutein and zeaxanthin are naturally found in dark green leafy vegetables. The average Western diet contains fewer than 3 mg of lutein and zeaxanthin daily. It would require about a bucket of green leafy vegetables to consume the approximately 20 mg of carotenoids per day needed to effectively repigment the macula. And some individuals despite a high consumption of green vegetables still suffer from AMD. This leads to the hypothesis that it is not enough to simply consume adequate amounts of lutein and zeaxanthin to enhance the production of meso-zeaxanthin but that the lutein and zeaxanthin must be properly absorbed by the body for such consumption to effectively result in any added health benefit. If taken as a supplement, meso-zeaxanthin is absorbed into the blood stream and effectively increases macular pigment levels. However, differences in serum level can be correlated to the type of formulation consumed.

Lutein Sorb® (U.S. patent application pending), a composition produced and marketed by Jarrow Formulas (Los Angeles, Calif., USA) for supporting superior eye health, combines free lutein with 3R,3′R zeaxanthin and meso-zeaxanthin as a dispersion laid onto a hydrophilic carrier consisting of marigold fractions (Tagetes species). However, this formulation is only available in capsule form.

There still exists the need for a nutritional supplement that combines dietary lutein and zeaxanthin with a combination of antioxidants, vitamins, minerals, and omega fatty acids in an easy once per day liquid formula that is readily assimilated.

### SUMMARY OF THE INVENTION

The present invention in one aspect provides a daily liquid supplement for a human subject for ocular and body health wherein the liquid supplement contains micronized critical nutrients to ensure optimal efficiency of absorption into the bloodstream.

In another aspect the present invention provides a daily liquid supplement for a human subject wherein the liquid supplement contains the essential macular carotenoids lutein, zeaxanthin, meso-zeaxanthin and astaxanthin.

In another aspect the present invention provides a daily liquid supplement in a micronized lipid based emulsion for efficient delivery of micronutrients to the human body.

In another aspect the present invention provides a daily liquid supplement wherein all the Ingredients are biocompatible with no redundancy or competitive absorption.

In another aspect the present invention provides a daily liquid supplement wherein the micronized ingredients remain in the body for extended periods of time.

In another aspect the present invention provides an emulsion for nutritionally supplementing a human subject, the emulsion comprising: an emulsion of sufficient quantity for nutritionally supplementing the human subject for one day; and a container including a vessel and a detachable lid, said vessel defining an enclosure for containing said emulsion, said detachable lid being attached to said vessel for sealing the enclosure and protecting said emulsion therein against oxidation, said detachable lid being detachable from said vessel once only for accessing said emulsion therein and for assuring non-oxidation of said emulsion; wherein further said emulsion including: a hydrophobic carotenoid selected from the group of macular pigment supplements consisting of lutein, zeaxanthin, meso-zeaxanthin and astaxanthin; a hydrophilic ocular antioxidant selected from the group consisting of bilberry fruit extract and alpha-lipoic acid; an aqueous solvent; a wetting agent for enhancing dispersal of said hydrophobic carotenoid within said aqueous solvent, said wetting agent being a water-dispersible food grade lysophospholipid; an emulsifier for emulsifying said hydrophobic carotenoid within said aqueous solvent, said emulsifier being a food grade hydrophilic non-ionic surfactant; a stabilizer for stabilizing the emulsion for at least several months, said stabilizer being a food grade natural product gum; an absence of more than a trace quantity of hydrophobic solvent; and an absence of more than a trace quantity of protein; said hydrophobic carotenoid and said hydrophilic ocular antioxidant being combined with said aqueous solvent, together with said wetting agent, said emulsifier, and said stabilizer by dispersal and emulsification for forming the emulsion for nutritionally supplementing macular pigments.

In another aspect the present invention provides an emulsion wherein the wetting agent is a partially hydrolyzed de-oiled lecithin. In still another aspect the present invention provides an emulsion wherein said wetting agent is the partially hydrolyzed de-oiled lecithin is derived from soy.

In another aspect the present invention provides an emulsion wherein the emulsifier is a polyethylene glycol sorbitan fatty acid mono-ester. In still another aspect the present invention provides an emulsion wherein said emulsifier is polysorbate 80.

In another aspect the present invention provides an emulsion wherein the stabilizer is selected from a group of natural gums consisting of pectin, xanthan, alginate, and guar gum. In still another aspect the present invention provides an emulsion wherein said stabilizer is xanthan gum.

In another aspect the present invention provides an emulsion wherein the aqueous solvent including water, a buffering agent, and a co-solvent for lowering interfacial tension of the aqueous phase and enhancing the activity of said wetting agent with respect to dispersal of said hydrophobic carotenoid within said aqueous solvent, the co-solvent being selected from the group consisting of an alcohol and a polyol. In still another aspect the present invention provides an emulsion wherein said co-solvent is glycerin. In still another aspect the present invention provides an emulsion wherein said buffering agent is a salt of citric acid. In still another aspect the present invention provides an emulsion wherein said water is purified by reverse osmosis.

In another aspect the present invention provides an emulsion further comprising a natural flavor and a sweetening agent. In still another aspect the present invention provides an emulsion lacking contact with an edible encapsulation material.

In another aspect the present invention provides an emulsion further comprising a water soluble nutritional supplement selected from the group consisting of acetyl-L carnitine, biotin, coenzyme Q10, folic acid, L-taurine, N-acetyl cysteine, quercetin, vitamin B1, vitamin B2, vitamin B3, vitamin B5, vitamin B6, vitamin B12, and vitamin C; said soluble nutritional supplement being admixed into the emulsion.

In another aspect the present invention provides an emulsion further comprising a hydrophobic nutritional supplement selected from the group consisting of lycopene, vitamin D3, and vitamin E; said hydrophobic nutritional supplement being admixed into the emulsion.

In another aspect the present invention provides an emulsion further comprising a mineral selected from salts of the group consisting of calcium, chromium, copper, magnesium manganese, molybdenum, potassium, selenium, and zinc; said mineral being admixed into the emulsion.

In another aspect the present invention provides an emulsion wherein the emulsion being characterized by having hydrophobic particles with an average diameter of 0.1-100 micrometers, preferably 80 micrometers or less, more preferably 75 micrometers or less, most preferably 60 micrometers or less.

In another aspect the present invention provides an emulsion wherein the emulsion being characterized by having an oil phase comprising at least 50% of a triglyceride having a fatty add chain length of 12 carbon atoms or great.

In another aspect the present invention provides an emulsion wherein the emulsion being characterized by having one or more additional oils such that the ratio of triglyceride to additional oil is preferably 1:0 to 1:1.

In another aspect the present invention provides an emulsion wherein the emulsion being characterized by having a total oil content including long chain triglycerides and addition oil of 0.01 to 70 wt % preferably 0.01 to 50 wt %, more preferably 0.01 to 40 wt %.

In another aspect the present invention provides an emulsion wherein the emulsion being characterized by having a food grade or pharmaceutical grade hydrophilic non-ionic surfactant with a hydrophilic-lipophilic balance (HLB) greater than 7.

In another aspect the present invention provides an emulsion wherein the emulsion being characterized by having a hydrophilic surfactant content of 0.1 to 15 wt %, preferably 1 to 10 wt %, more preferably 3 to 7 wt %.

In another aspect the present invention provides an emulsion wherein the emulsion being characterized by having a food grade co-surfactant content of 0.1 to 15 wt %. Preferably the co-surfactant is present in a ratio relative to the hydrophilic non-ionic surfactant of 0:1 to 2:1, more preferably 0:1 to 1.3:1 and most preferably 0.5:1 to 1.3:1.

In another aspect the present invention provides an emulsion wherein the emulsion being characterized by having a water content of 50 to 100 wt %, preferably 40 to 99.99 wt %, more preferably 30 to 99.90 wt %.

In another aspect the present invention provides an emulsion wherein the emulsion being characterized by having a co-solvent content of 0 to 70 wt %, preferably 0 to 50 wt %, more preferably 15 to 45 wt %.

In another aspect the present invention provides an emulsion wherein the emulsion being characterized by having an active component content of 0.01 to 50 preferably 0.01 to 10 wt %.

In another aspect the present invention provides an emulsion wherein the emulsion being characterized by having an additive component of 0 to 50 wt %, preferably 0 to 25 wt %, more preferably 0 to 10 wt %.

In another aspect the present invention provides a method of formulating a nutritional supplement for the macular pigments of a human subject, the method comprising:

a) preparing a first Preblend mixture;

b) preparing a second Preblend mixture;

c) slowly adding the first Preblend to the second Preblend until the pH specification is achieved, preferably 3.9-4.3 for stability of the preservative system and taste; and

d) mixing with high sheer for a minimum of 6 hrs,

wherein an emulsion is formed.

In another aspect the present invention provides a kit comprising: a box; a plurality of first containers, each container containing a once daily dose of an emulsion for supplementing macular carotenoids, the emulsion being an aqueous/hydrophobic emulsion including carotenoids, bilberry fruit extract, and alpha-lipoic acid, each of said containers being sealed for preventing oxidation of the medicament therein; at least one second container containing a plurality of twice daily doses of omega fatty adds; said first and second containers being packed as a kit within said box for supplying a plurality of once daily doses of the emulsion in combination with but separate from the omega fatty acids.

### Nanoemulsion

The term “nanoemulsion” refers to oil-in-water emulsions in which the oil droplets are ultra-small having a diameter of 100 nm or less, preferably 80 nm or less, more preferably 75 nm or less, most preferably 60 nm or less. The droplet size is the Z-average or Intensity weighted average size as measured by dynamic light scattering (also known as photon correlation spectroscopy).

### Oil Phase

The oil phase comprises at least 50 volume % of a triglyceride having a fatty add chain length of 12 carbon atoms or greater. The triglyceride can be a liquid or solid fat of animal, vegetable, algal or synthetic origin which is preferably food grade having the following general formula:

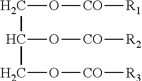

in which R₁, R₂ and R₃ are independently selected from saturated and unsaturated fatty acid residues (unbranched and branched) with chain lengths of C₁₂ or greater, preferably C₁₂-C₂₄. Most preferably, the chain lengths are between C₁₆-C₂₂, i.e. long chain triglycerides. Long chain triglycerides, preferably having some degree of unsaturation have been shown to provide positive nutritional benefits and are considerably more stable against Ostwald ripening.

Examples of long chain triglycerides include those of animal origin such as fish oil, cod liver oil, blubber, lard, tallow, schmaltz, and butter fat, vegetable origin such as canola oil, castor oil, cocoa butter, coconut oil, coffee seed oil, corn oil, cotton seed oil, evening primrose oil, grapeseed oil, flax seed oil, menhaden oil, mustard seed oil, olive oil, palm oil, palm kernel oil, peanut oil, poppy seed oil, rapeseed oil, rice bran oil, safflower oil, sesame oil, soybean oil, sunflower oil, palm kernel oil, hazelnut oil, sesame oil and wheat germ oil. Examples of long chain triglycerides of algal origin such as vegetable oil and synthetic triglycerides, fractionated triglycerides, modified triglycerides, hydrogenated triglycerides or partially hydrogenated and mixtures of triglycerides are also included.

The nanoemulsion may contain one or more additional oils such as short chain triglycerides for example triacetin, tributyrin, tricapylrin and miglyol. Additionally included are mineral oils, for example alkane oils such as decane, tetradecane, hexadecane and octadecane, as well as flavor oils for example limonene, mandarin oil orange oil, lemon oil, lime oil or other citrus oils, peppermint oil, peach oil, vanilla flavor oil and vanillin; and aromatic oils for example peppermint, tea tree oil, eucalyptus oil, mentha arvensis, cedarwood oil, spearmint, orange oil, lemin oil and clove.

The ratio of triglyceride to additional oil is preferably 1:0 to 1:1.

The total amount of oil in the nanoemulsion including long chain triglyceride and additional oil if present may be 0.01 to 70 wt %, preferably 0.01 to 50 wt %, more preferably 0.01 to 40 wt %.

### Hydrophilic Non-Ionic Surfactant

The hydrophilic non-ionic surfactant has a hydrophilic-lipophilic balance (HLB) greater than 7 and is preferably a food grade or pharmaceutical grade hydrophilic surfactant such as polysorbates (polyethylene glycol sorbitan fatty acid esters), polyethylene glycol alkyl ethers, sugar esters, polyethoxylated fatty adds, polyoxyethylene-polyoxypropylene block co-polymers (Pluronics), polyethylene glycol alkyl phenol surfactants, citric acid esters of monoglycerides, polyglycerol esters, polyethoxylated fatty acid diesters, PEG-fatty acid mono and diesters, polyethylene glycol glycerol fatty acid esters and alcohol oil transesters or mixtures thereof.

Suitable non-ionic surfactants include: polysorbates for example polyethoxyethylene sorbitan monoesters, including polyoxyethylene sorbitan monolaurate (Tween 20), polyoxyethylene sorbitan monopalmitate (Tween 40), polyoxyethylene sorbitan monostearate (Tween 60), polyoxyethylene sorbitan tristearate (Tween 65) and polyoxyethylene sorbitan mono-oleate (Tween 80); sugar surfactants for example sucrose monopalmitate, sucrose monolaurate, sucrose distearate 3 Crodesta F-10, sucrose distearate, monostearate Crodesta F-110, sucrose dipalmitate, sucrose monostearate Crodesta F-160, sucrose monopalmitate, sucrose monolaurate and saccharose monolaurate; polyoxyethylene-polyoxypropylene block co-polymers which are available under various trade names including Synperonic PE series (ICI), Pluronic® series (BASF), Emkalyx, Lutrol (BASF), Supronic, Monolan, Pluracare and Plurodac. The polyoxyethylene-polyoxypropylene block co-polymers are also known as “polyoxamers” and have the general formula:

HO(C₂H₄O)_{A}(C₃H₆O)_{B}(C₂H₄O)_{A}H

In which A and B denote the number of polyoxyethylene and polyoxypropylene units, respectively. Polyoxamers when A is 1-100 and B is 1-100 and combinations thereof are suitable for use in the nanoemulsions of the present invention. The amount of hydrophilic surfactant in the nanoemulsion may be 0.1 to 15 wt %, preferably 1 to 10 wt %, more preferably 3 to 7 wt %.

### Co-Surfactant

The nanoemulsion may also contain a co-surfactant which s preferably a surfactant that acts synergistically with the hydrophilic non-ionic surfactant to alter the Interfacial curvature. This lowers interfacial tension, permitting easier emulsion formation. Preferably the co-surfactant is food grade or pharmaceutical grade.

Suitable food grade co-surfactants include: sorbitan fatty acid esters such as sorbitan monolaurate (Span 20), sorbitan monopalmitate (Span 40), sorbitan tristearate (Span 65), sorbitan monostearate (Span 60), sorbitan monooleate (Span-80) and sorbitan trioleate (Span-85); phospholipids such as egg/soy lecithin for example epikuron, topcithin, leciprime, lecisoy, emulfluid, emulpur, metarin, emultop, lecigran, lecimulthin, ovothin lyso egg/soy lecithin, hydroxylated lecithin lysophosphatidylcholine, cardiolipin, sphingomyelin, phosphatidylcholine, phosphatidyl ethanolamine, phosphatidic acid, phosphatidyl glycerol, phosphatidyl serine and mixtures of phospholipids with other surfactants; and ionic surfactants such as sodium stearoyl lactylate and calcium stearoyl lactylate.

The amount of co-surfactant in the nanoemulsion may be 0.1 to 15 wt %. Preferably the co-surfactant is present in a ratio relative to the hydrophilic non-ionic surfactant of 0:1 to 2:1, more preferably 0:1 to 1.3:1 and most preferably 0.5:1 to 1.3:1.

### Aqueous Phase

The aqueous phase can be either purified or ultrapure water, saline or buffered saline. The balance of water after the inclusion of all other formulation components in the nanoemulsion may be 50 to 100 wt %, preferably 40 to 99.99 wt %, more preferably 30 to 99.90 wt %.

### Co-Solvent

In a preferred embodiment, the nanoemulsion also contains a co-solvent. The co-solvent lowers the interfacial tension of the aqueous phase which thereby enables the formation of smaller emulsion droplet sizes.

Suitable co-solvents include C1-C10 alcohols such as methanol, ethanol, propanol, butanol, pentanol, hexanol, heptanol, octanol, nonanol and decanol; polyols such as glycerol, 1,2 propandiol, 1,3 propandiol, polyethylene glycol and polypropylene glycol; and long chain fatty alcohols. Preferably, the co-solvent is an alcohol or a polyol, more preferably glycerin.

The amount of co-solvent in the nanoemulsion may be 0 to 70 wt %, preferably 0 to 50 wt %, more preferably 15 to 45 wt %.

### Active Component

The active component is any component that is an oil, oil-soluble, partitions to an oil phase, poorly soluble in oil and water or soluble or capable of being dispersed at an Interface which imparts either a color, aroma, flavor, antimicrobial effect, beautification effect, health promoting effect, disease prevention effect or technique, or disease curing effect to the nanoemulsion.

The active components may be food or beverage ingredients such as food supplements, food additives, aromas, aromatic oils, colors, flavors and sweeteners; cosmetics; pharmaceuticals such as medicaments, peptides, proteins and carbohydrates; nutraceuticals; phytochemicals; vitamins; essential polyunsaturated fatty acids; plant extracts; agrichemicals such as pesticides and herbicides; textiles; polymers; and chemicals.

Suitable active components include: phytochemicals such as polyphenols (e.g., catechin, epicatechin, epicatechin gallate, quercetin and resveratrol), carotenoids (e.g., lycopene, lutein, lutein esters, β-carotene, retinyl, retinyl palmitate and zeaxanthin), ubiquinone (CoQ10) and phytosterols; vitamins such as vitamin A (e.g., retinol and retinol palmitate), Vitamin D (e.g., calciferol), vitamin E (e.g., tocopherol, tocopherol acetate and tocopherol palmitate), vitamin K (e.g., K1-phylloquinone and K2-menaquinone); essential polyunsaturated fatty acids such as linoleic acid, alpha-linolenic acid, eicosapentaenoic acid and docosahexaenoic acid; flavours such as natural flavour oils for example citrus oil, limonene, mandarin oil orange oil, lemon oil, lime oil, peppermint oil, peach oil, vanilla flavour oil and vanillin or synthetic flavoring materials for example hexyl alcohol, ethyl laurate, apple flavoring oil, strawberry flavoring oil, benzaldehyde, cinnamic aldehyde, paprika flavoring oil, citronellyl butyrate, phenyl ethyl acetate, ethyl propionate, ethyl decanoate, ethyl butyrate, ethyl hexanoate, brandy flavoring oil, hexyl aldehyde, blackberry flavoring oil, phelandrene, blueberry flavoring oil, honey flavoring, oil, nerol, licorice flavoring oil, maple flavoring oil, ethyl caprylate and watermelon flavoring oil; and aromatic oils such as peppermint, tea tree oil, eucalyptus oil, mentha arvensis, cedarwood oil, spearmint, orange oil lemin oil and clove.

The amount of active component in the nanoemulsion may be 0.01 to 50 preferably 0.01 to 10 wt %.

### Additives

The nanoemulsion may contain additives such as stabilizers, antioxidants, preservatives, buffering agents, charge inducing agents, weighting agents polymers and proteins. Stabilizers can be pH modifying agents, anti-creaming or anti-foaming agents or agents which impart stability to the nanoemulsion. Examples of stabilizers include sodium oleate, glycerine, xylitol, sorbitol, ascorbic acid, citric add and sodium edetate. Antioxidants include carotenoids, for example alpha-tocopherol or its derivatives, which are members of the Vitamin E family, β-carotene, lutein, lycopene, ascorbic acid, trolox, β-carotene, polyphenols such as catechin, epicatechin, epicatechin gallate, quercetin, resveratrol, ascorbyl palmitate and butylated hydroxytoluene (BHT). Buffering agents include sodium phosphate, citric acid, formic acid and ascorbic acid. Examples of charge inducing agents include sodium deoxycholate, sodium lauryl sulfate, deoxycholic acid, stearylamine, oleylamine, chitosan and cetyltriethylammonium bromide. Weighting agents include brominated vegetable oils. Examples of polymers and proteins include hydrocolloids such as guar gum, pectin, xanthan and alginate.

The amount of additive in the nanoemulsion may be 0 to 50 wt %, preferably 0 to 25 wt %, more preferably 0 to 10 wt %.

### BRIEF DESCRIPTION OF THE DRAWINGS

FIG. 1 illustrates the increase in patient MPOD following administration of the formulation of the present invention.

FIG. 2 illustrates the efficiency of absorbency of the formulation of the present invention versus solid form supplements.

FIG. 3 illustrates the closed kit of the present invention.

FIG. 4 illustrates the open kit and contents of said kit of the present invention.

### DETAILED DESCRIPTION OF THE DRAWINGS

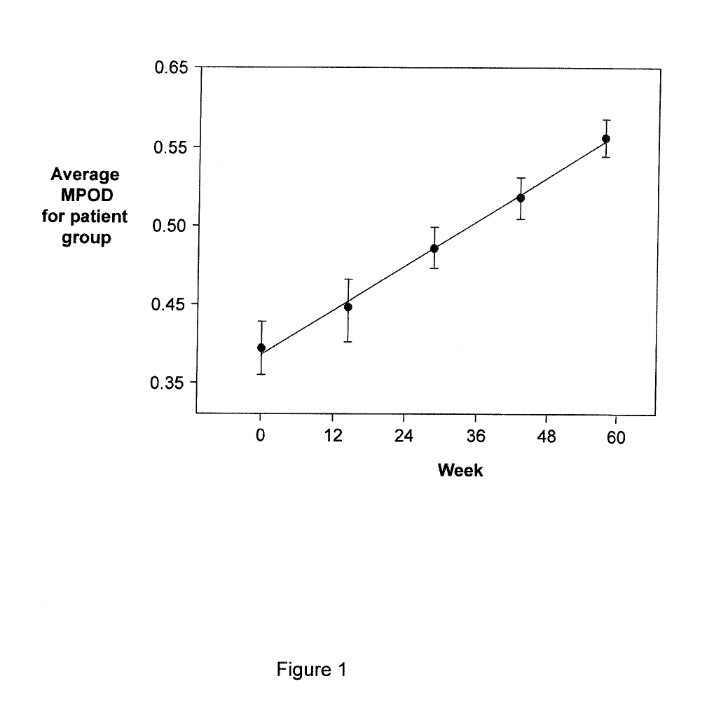

FIG. 1 is a graph showing the average increase in MPOD of 872 patients taking the formulation of the present invention as measured using the MAPCATsf at weeks 14, 30, 42 and 60 of administration.

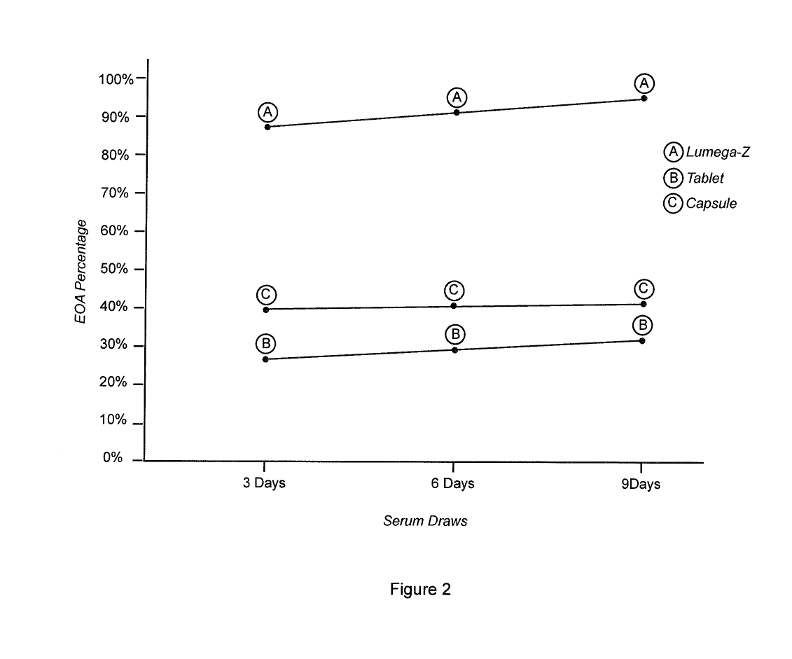

FIG. 2 is a graph showing the percentage of micronutrient absorption of the liquid formulation of the present invention in patient serum collected on days 3, 6 and 9 of administration compared to the percentage absorption of solid form supplements, i.e. tablets and capsules.

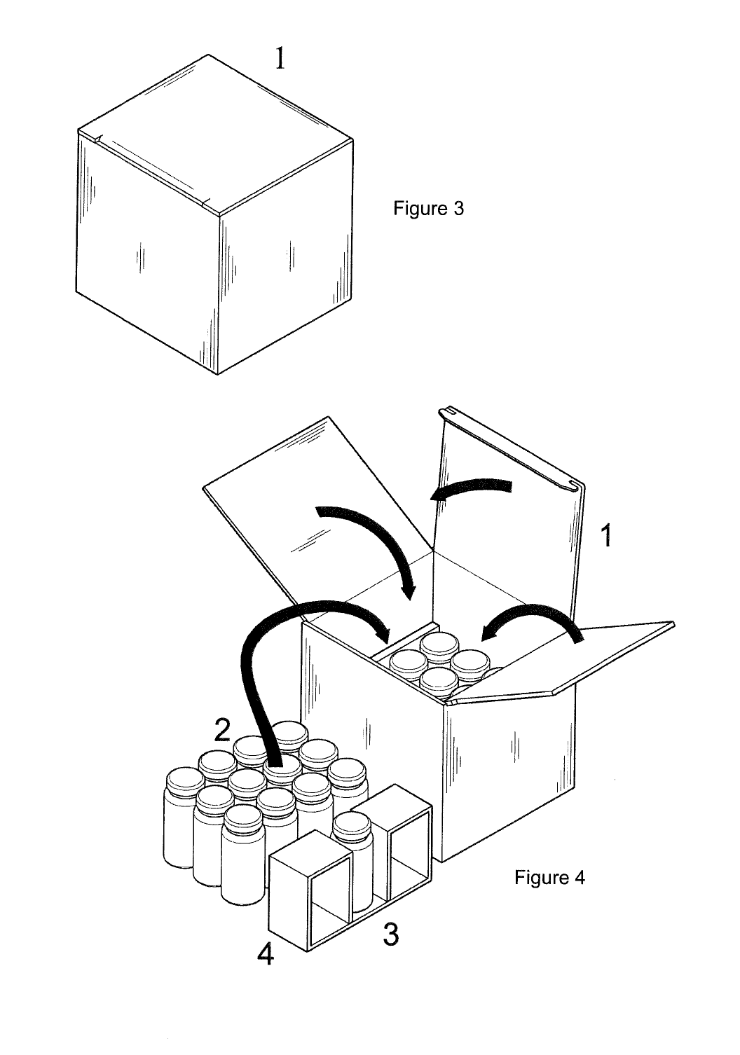

FIG. 3 illustrates the kit 1 containing the packaged emulsion of the present invention.

FIG. 4 illustrates the kit 1 of FIG. 3, having 28 sealed bottles 2 filled with the emulsion to be administered once daily, one bottle (container) 3 containing at least 28 omega fatty acid capsules, two spacers 4, and an instruction/Information insert (shown already inside box). The kit contains a 28 day supply of the emulsion and the omega fatty acid capsules, i.e., a 4 week supply.

### DETAILED DESCRIPTION OF THE INVENTION

The following examples are intended to illustrate the invention by way of example only, and are not intended to limit the scope of the invention.

### Example 1

### Determination of Daily Dosage

The appropriate dosage of the emulsion for daily administration to a patient is determined by first obtaining a baseline measurement of the patient's Macular Pigment Optical Density (MPOD) using the MAPCATsf. The baseline measurement obtained will determine the correct dosage of the emulsion to administer to a specific patient resulting in enhanced assayable macular pigment levels.

### Example 2

### Emulsion Preparation

In carrying out the present invention, the total daily serving size in weight is 67239.804 mg/serving comprised of approximately 48685.000 mg Water (Reverse Osmosis), 315.000 mg Water (Reverse Osmosis) and 200.000 mg Xanthan Gum (Clear Gel).

The packaged emulsion of the present invention is formulated according to the following protocol:

### Preblend A:

1. Combine Water 87 wt % and Glycerin 12.6 wt % slowly. Next add Xanthan Gum 0.5 wt % under high speed mixing to make a slurry. Add polysorbate 80 3.7 wt %.
2. Add Vitamin E (mixed tocopherols, including gamma-tocopherol), Vitamin D, Lumega Carotenoid Blend, Lycopene, Quercetin, Astaxanthin and Lecithin one at a time to Preblend A. Mix with high sheer for 20 minutes or until homogeneous.
3. Add remaining fat solubles one at a time
4. Add water solubles one at a time

### Preblend B:

1. Combine Water, Citric Acid 0.5 wt % and mix slowly until there are no visible solids.

Slowly add Preblend A to Preblend B until the pH specification is achieved, preferably 3.9-4.3 for stability of the preservative system and taste. Mix with high sheer for a minimum of 6 hrs prior to filling.

### Example 3

Table 1 shows an example LUMEGA-Z emulsion formulation.

TABLE 1
LUMEGA-Z Formula Specification:

| | |
|---|---|
| Amount per | % Daily |
| serving | Value |

| | | | |
|---|---|---|---|
| Vitamin C | 500 | mg | 833% |
| Thiamin | 1.5 | mg | 100% |
| Riboflavin | 1.7 | mg | 100% |
| Niacin | 20 | mg | 100% |
| Vitamin B6 | 10 | mg | 500% |
| Folate | 800 | mcg | 200% |
| Vitamin B12 | 1000 | mcg | 16667% |
| Vitamin D3 | 2000 | IU | 500% |
| Vitamin E | 200 | IU | 665% |
| Biotin | 100 | mcg | 33% |
| Pantothenic Acid | 10 | mg | 100% |
| Calcium | 250 | mg | 25% |
| Magnesium | 100 | mg | 25% |
| Zinc | 25 | mg | 167% |
| Selenium | 70 | mcg | 100% |
| Copper | 3 | mg | 150% |
| Manganese | 2 | mg | 100% |
| Chromium | 120 | mcg | 100% |
| Molybdenum | 75 | mcg | 100% |
| NAC (N-acetyl-cysteine) | 500 | mg | * |
| Proprietary Ocular Antioxidant Blend | 200 | mg | * |
| (billberry fruit extract 4:1, alpha- | | | |
| kipoic acid) | | | |
| Acetyl-L-Carnitine | 500 | mg | * |
| Taurine | 500 | mg | * |
| Quercetin | 100 | mg | * |
| CoQ10 | 50 | mg | * |
| Lycopene | 500 | mcg | * |
| Lutein | 15 | mg | * |
| Zeaxanthin | 3 | mg | * |
| Meso-Zeaxanthin | 10 | mg | * |
| Astaxanthin | 1000 | mcg | * |

* Daily value not established.

### Example 4

### Results of Lumega-Z Administration to Human Subject

The total number of patients that participated in the study was 872. Patients then returned after weeks 14, 30, 42 and 60 of Lumega-Z administration for retesting on the MAPCATsf.

The results dearly showed that the formula was effective on all patients, without exception, by increasing patient MPOD (FIG. 1). There were few patients who only showed an Increase of 15-18%, and it is now presumed that these individuals may have more difficulty absorbing the carotenoids or they were not taking the Lumega-Z every day as indicated. The remaining patients showed increases in MPOD ranging from 20% to 30%.

### Example 5

Analysis of Efficiency of Absorption for Lumega-Z v. Solid Form Supplements

The efficiency of absorption (EOA) of lipid based micronized liquid, Lumega-Z, was compared with solid form supplements. Two products were compared to the Lumega-Z formulation: 1) a widely used multi-vitamin in tablet form (tablet); and 2) a commonly prescribed over the counter products used for patients diagnosed with macular degeneration in capsule form (capsule).

The total number of subjects for the analysis was twelve: four subjects received Lumega-Z; four subjects received the multi-vitamin tablets; and four subjects received the AMD capsule. Serum samples were collected from each subject on day 3, 6 and 9 and concentrations of key micronutrients represented in all administered forms were quantified by high performance liquid chromatography (HPLC).

The results surprisingly showed that the subjects taking Lumega-Z demonstrated an EOA of ±90% compared to ±32% and ±39% for the tablet and capsule, respectively (FIG. 2). These data show that when taken as a dietary supplement Lumega-Z may significantly increase patient MPOD over time resulting in the prevention of macular degeneration and/or the correction of atypical macular pigment distribution. These data also show that the formulation of the present invention is superior to similar solid form supplements currently available and is more efficient at delivering the necessary micronutrients to the human body.

It will be appreciated that details of the foregoing embodiments, given for purposes of illustration, are not to be construed as limiting the scope of this invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed is:

1. A lipophilic formulation for nutritionally supplementing a human subject, the formulation comprising:
a) hydrophobic carotenoids lutein, zeaxanthin, and meso-zeaxanthin;
b) at least one hydrophilic ocular antioxidant selected from the group consisting of bilberry fruit extract and alpha-lipoic acid;
c) an aqueous solvent;
d) a wetting agent for enhancing dispersal of said hydrophobic carotenoid within said aqueous solvent, said wetting agent being a water-dispersible food grade lysophospholipid;
e) an emulsifier for emulsifying said hydrophobic carotenoid within said aqueous solvent, said emulsifier being a food grade hydrophilic non-ionic surfactant;
f) a stabilizer for stabilizing the emulsion for at least several months, said stabilizer being a food grade natural product gum;
g) an absence of more than a trace quantity of hydrophobic solvent;
h) an absence of more than a trace quantity of protein; and
i) an oil phase comprising at least 50% of a triglyceride having a fatty acid chain length of 12 carbon atoms or greater;
said hydrophobic carotenoid and said hydrophilic ocular antioxidant being combined with said aqueous solvent, together with said wetting agent, said emulsifier, said stabilizer and said oil phase by dispersal and emulsification for forming an emulsion for nutritionally supplementing macular pigments.

2. The formulation of claim 1 wherein the triglyceride is a long chain triglyceride selected from at least one of the group consisting of fish oil, cod liver oil, blubber, lard, tallow, schmaltz, butter fat, canola oil, castor oil, cocoa butter, coconut oil, coffee see oil, corn oil, cotton seed oil, evening primrose oil, grapeseed oil, flax seed oil, menhaden oil, mustard seed oil, olive oil, palm oil, palm kernel oil, peanut oil, poppy seed oil, rapeseed oil, rice bran oil, safflower oil, sesame oil, soybean oil, hazelnut oil and wheat germ oil.

3. The formulation of claim 2 wherein said wetting agent is a partially hydrolyzed de-oiled lecithin.

4. The formulation of claim 3 wherein the partially hydrolyzed de-oiled lecithin is derived from soy.

5. The formulation of claim 2 wherein said emulsifier is a polyethylene glycol sorbitan fatty acid mono-ester.

6. The formulation of claim 5 wherein said emulsifier is polysorbate 80.

7. The formulation of claim 2 wherein said stabilizer is selected from the group of natural gums consisting of pectin, xanthan, alginate, and guar gum.

8. The formulation of claim 7 wherein said stabilizer is xanthan gum.

9. The formulation of claim 2 wherein said aqueous solvent including water, a buffering agent, and a co-solvent for lowering interfacial tension of the aqueous phase and enhancing the activity of said wetting agent with respect to dispersal of said hydrophobic carotenoid within said aqueous solvent, the co-solvent being selected from the group consisting of an alcohol and a polyol.

10. The formulation of claim 9 wherein the co-solvent is glycerin.

11. The formulation of claim 9 wherein the buffering agent is a salt of citric acid.

12. The formulation of claim 10 wherein the water is purified by reverse osmosis.

13. The formulation of claim 2 further comprising a natural flavor and a sweetening agent.

14. The formulation of claim 2 further comprising a water soluble nutritional supplement selected from the group consisting of acetyl-L carnitine, biotin, coenzyme Q10, folic acid, L-taurine, N-acetyl cysteine, quercetin, vitamin B1, vitamin B2, vitamin B3, vitamin B5, vitamin B6, vitamin B12, and vitamin C, said soluble nutritional supplement being admixed into the emulsion.

15. The formulation of claim 2 further comprising a hydrophobic nutritional supplement selected from the group consisting of lycopene, vitamin D3, and vitamin E, said hydrophobic nutritional supplement being admixed into the emulsion.

16. The formulation of claim 2 further comprising a mineral selected from salts of the group consisting of calcium, chromium, copper, magnesium manganese, molybdenum, potassium, selenium, and zinc; said mineral being admixed into the emulsion.

17. The formulation of claim 2 wherein the emulsion being characterized by having hydrophobic particles with an average diameter of 0.1-100 micrometers.

18. A method of formulating a nutritional supplement for the macular pigments of a human subject, the method comprising:
a) preparing a first Preblend mixture;
b) preparing a second Preblend mixture;
c) slowly adding the first Preblend to the second Preblend until the pH specification is achieved, preferably 3.9-4.3 for stability of the preservative system and taste; and
d) mixing with high sheer for a minimum of 6 hrs,
wherein an emulsion is formed.

19. The method of claim 18, wherein the first Preblend mixture consists of a mixture prepared according to the following steps:
1) combining Water 87 wt % and Glycerin 12.6 wt % slowly;
2) adding Xanthan Gum 0.5 wt % to the product of step 1) under high speed mixing to make a slurry;
3) adding polysorbate 80 3.7 wt % to the slurry of step 2);
4) adding Vitamin E (mixed tocopherols, including gamma-tocopherol), Vitamin D, Lumega Carotenoid Blend, Lycopene, Quercetin, Astaxanthin and Lecithin one at a time to the product of step 3), mixing with high sheer for 20 minutes or until homogeneous;
5) adding remaining fat solubles one at a time; and
6) adding water solubles one at a time.

20. The method of claim 18, wherein the second Preblend mixture consists of a mixture prepared by combining Water and Citric Acid 0.5 wt % and further mixing slowly until there are no visible solids.

21. A kit comprising:
(a) a box; the box further comprising a plurality of first containers, each container containing a once daily dose of a medicament for supplementing macular carotenoids, the medicament being an aqueous/hydrophobic emulsion including carotenoids, bilberry fruit extract, and alpha-lipoic add, each of said containers being sealed for preventing oxidation of the medicament therein; and
(b) at least one second container containing a plurality of once daily doses of omega fatty acids, said first and second containers being packed as a kit within said box for supplying a plurality of once daily doses of the medicament in combination with but separate from the omega fatty acids.
